(19) United States
(12) Patent Application Publication
(10) Pub. No.: US 2016/0176778 A1
(43) Pub. Date: Jun. 23, 2016

(54) PROCESS FOR CONVERSION OF LIGHT ALIPHATIC HYDROCARBONS TO AROMATICS

(71) Applicant: UOP LLC, Des Plaines, IL (US)

(72) Inventors: Deng-Yang Jan, Elk Grove Village, IL (US); Pelin Cox, Des Plaines, IL (US)

(21) Appl. No.: 14/574,293

(22) Filed: Dec. 17, 2014

(51) Int. Cl. C07C 2/76

(52) U.S. Cl. CPC C07C 2/76; C07C 2529/068; C07C 2529/06; C07C 2529/076

(57) ABSTRACT

A process is disclosed for the aromatization of light aliphatic hydrocarbons, such as propane, into aromatic hydrocarbons. The process provides increased aromatics production, decreasing methane and ethane production, coke fouling and decreasing heavy aromatics. This improvement for the aromatization of light aliphatic hydrocarbons is achieved by introducing heavier of the light alphatic hydrocarbons in the feed to the lag reactors.

### FIELD

The present subject matter relates generally to methods for hydrocarbon conversion. More specifically, the present subject matter relates to methods for a catalytic process referred to as dehydrocyclodimerization wherein two or more molecules of a light aliphatic hydrocarbon, such as propane or propylene, are joined together to form a product aromatic hydrocarbon.

### BACKGROUND

Dehydrocyclo-oligomerization is a process in which aliphatic hydrocarbons are reacted over a catalyst to produce aromatics, hydrogen and certain byproducts. This process is distinct from more conventional reforming where C₆ and higher carbon number reactants, primarily paraffins and naphthenes, are converted to aromatics. The aromatics produced by conventional reforming contain the same or a lesser number of carbon atoms per molecule than the reactants from which they were formed, indicating the absence of reactant oligomerization reactions. In contrast, the dehydrocyclo-oligomerization reaction results in an aromatic product that typically contains more carbon atoms per molecule than the reactants, thus indicating that the oligomerization reaction is an important step in the dehydrocyclo-oligomerization process. Typically, the dehydrocyclo-oligomerization reaction is carried out at temperatures in excess of 260° C. using dual functional catalysts containing acidic and dehydrogenation components.

Aromatics, hydrogen, a C₄₊ nonaromatics byproduct, and a light ends byproduct are all products of the dehydrocyclo-oligomerization process. The aromatics are the desired product of the reaction as they can be utilized as gasoline blending components or for the production of petrochemicals. Hydrogen is also a desirable product of the process. The hydrogen can be efficiently utilized in hydrogen consuming refinery processes such as hydrotreating or hydrocracking processes. The least desirable product of the dehydrocyclo-oligomerization process is light ends byproducts. The light ends byproducts consist primarily of C₁ and C₂ hydrocarbons produced as a result of the cracking side reactions.

Traditionally, the dehydrocyclodimerization process includes a combined reactor feed having both C₃ and C₄ and recycled light paraffin feed components. While increasing the C₄ content in the feed increases yields, the pyrolytic coking becomes much more severe. Consequently, the on-stream efficiency is impacted adversely. Pyrolytic coking in the reactor internals is due to the formation of di-olefins mainly butadiene from n-butane and n-butene in the feed stream. Pyrolytic coking is most severe in the lead reactor due to lower hydrogen partial pressure and low aromatic components. Furthermore, reactivity of light aliphatic hydrocarbon increases with increasing carbon numbers. Therefore, conversions of butane takes place at significantly lower temperatures than propane, invariably a significant amounts of propane is not converted in C4 rich feed. Consequently, propane conversion is limited and a significant propane recycle is required.

### SUMMARY

The claimed subject matter includes a process of producing aromatic hydrocarbons including passing a first light aliphatic hydrocarbon feed stream rich in C₂-C₃ hydrocarbons to a first reaction zone having a first catalyst to form a first reaction zone effluent. The method further includes passing the first reaction zone effluent and a second light aliphatic hydrocarbon feed stream rich in C₃-C₅ hydrocarbons to second reaction zone comprising a second catalyst to form second reaction zone effluent.

This method does not introduce a C₄ rich feed into the lead reactor, when C₂-C₃ are present in the feed, but only to the lag reactors. By introducing a C₄ rich feed into lagging reactors, where both H₂ and aromatics are present, it greatly diverts the propensity to form butadiene, therefore reducing coke fouling. Furthermore, reducing contact times for C₄ conversions greatly mitigate the heavy aromatics formation, thus yields higher desirable aromatics products and mitigating the heavy fouling in the lag reactors. It is further recognized that introducing a C₃ rich feed into the lead reactor allows for more severe operating temperatures and lower pressures to drive the aromatics yields with no concerns of generating coking and thus fouling. This also minimizes the production of excessive light ends including C₁ and C₂ derived from the cracking of C₄ or heavier.

Additional objectives, advantages and novel features of the examples will be set forth in part in the description which follows, and in part will become apparent to those skilled in the art upon examination of the following description and the accompanying drawings or may be learned by production or operation of the examples. The objectives and advantages of the concepts may be realized and attained by means of the methodologies, instrumentalities and combinations particularly pointed out in the appended claims.

### DEFINITIONS

As used herein, the term “dehydrocyclodimerization” is also referred to as aromatization of light paraffins. Within the subject disclosure, dehydrocyclodimerization and aromatization of light hydrocarbons are used interchangeably.

As used herein, the term “stream”, “feed”, “product”, “part” or “portion” can include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. The stream can also include aromatic and non-aromatic hydrocarbons. Moreover, the hydrocarbon molecules may be abbreviated C₁, C₂, C₃, Cn where “n” represents the number of carbon atoms in the one or more hydrocarbon molecules or the abbreviation may be used as an adjective for, e.g., non-aromatics or compounds. Similarly, aromatic compounds may be abbreviated A₆, A₇, A₈, An where “n” represents the number of carbon atoms in the one or more aromatic molecules. Furthermore, a superscript “+” or “−” may be used with an abbreviated one or more hydrocarbons notation, e.g., C₃₊ or C₃₋₃, which is inclusive of the abbreviated one or more hydrocarbons. As an example, the abbreviation “C₃₊” means one or more hydrocarbon molecules of three or more carbon atoms.

As used herein, the term “zone” can refer to an area including one or more equipment items and/or one or more sub-zones. Equipment items can include, but are not limited to, one or more reactors or reactor vessels, separation vessels, distillation towers, heaters, exchangers, pipes, pumps, compressors, and controllers. Additionally, an equipment item, such as a reactor, dryer, or vessel, can further include one or more zones or sub-zones.

As used herein, the term “rich” can mean an amount of at least generally 50%, and preferably 70%, by mole, of a compound or class of compounds in a stream.

As used herein, the term “substantially” can mean an amount of at least generally 80%, preferably 90%, and optimally 99%, by mole or weight, of a compound or class of compounds in a stream.

As used herein, the term “active metal” can include metals selected from IUPAC Groups that include 6, 7, 8, 9, 10, and 13 such as chromium, molybdenum, tungsten, rhenium, platinum, palladium, rhodium, iridium, ruthenium, osmium, copper, zinc, silver, gallium, and indium.

As used herein, the term “modifier metal” can include metals selected from IUPAC Groups that include 11-17. The IUPAC Group 11 trough 17 includes without limitation sulfur, gold, tin, germanium, and lead.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a schematic depiction of an exemplary aromatic production process in accordance with various embodiments for the production of aromatics.

FIG. 2 is a schematic depiction of another exemplary aromatic production process in accordance with various embodiments for the production of aromatics.

FIG. 3 is a schematic depiction of yet another exemplary aromatic production process in accordance with various embodiments for the production of aromatics.

FIG. 4 is a schematic depiction of another exemplary aromatic production process in accordance with various embodiments for the production of aromatics.

### DETAILED DESCRIPTION

The following detailed description is merely exemplary in nature and is not intended to limit the application and uses of the embodiment described. Furthermore, there is no intention to be bound by any theory presented in the preceding background or the following detailed description.

The various embodiments described herein relate to methods for hydrocarbon conversion. More specifically, the present subject matter relates to methods for a catalytic process referred to as dehydrocyclodimerization wherein two or more molecules of a light aliphatic hydrocarbon, such as, for example, propane or propylene, are joined together to form an aromatic hydrocarbon product. The basic utility of the process is the conversion of the low cost and highly available light aliphatic hydrocarbons, for example, C₃ and C₄ hydrocarbons, into more valuable aromatic hydrocarbons and hydrogen. This may be desired simply to upgrade the value of the hydrocarbons. It may also be desired to capitalize on a large supply of the C₃ and C₄ hydrocarbons or to fulfill a need for the aromatic hydrocarbons. The aromatic hydrocarbons produced can be used for various applications, including in the production of a wide range of petrochemicals, including benzene, a widely used basic feed hydrocarbon chemicals. The product aromatic hydrocarbons are also useful as blending components in high octane number motor fuels.

The feed composition for dehydrocyclodimerization process can vary depend on the compositions of light aliphatic hydrocarbon sources. In accordance with one aspect, the feed compounds to a dehydrocyclodimerization process include light aliphatic hydrocarbons having from 2 to 4 carbon atoms per molecule. The feed stream may comprise only one of C₂, C₃, and C₄ compounds or a mixture of two or more of these compounds. In one example, the feed compounds include one or more of propane, propylene, butanes, and the butylenes. The feed stream to the process may also contain some C₅ hydrocarbons. In one approach, the concentration of C₅ hydrocarbons in the feed stream to a dehydrocyclodimerization process is held to a maximum practical level, preferably below 5 mole percent. By one aspect, the products of the process include C₆-plus aromatic hydrocarbons. In addition to the desired C₆-plus aromatic hydrocarbons, some nonaromatic C₆-plus hydrocarbons may be produced, even from saturate feeds. When processing a feed made up of propane and/or butanes, the a large portion of the C₆-plus product hydrocarbons will be benzene, toluene, and the various xylene isomers. A small amount of C₉-plus aromatics may also be produced.

In accordance with one aspect, the process includes increasing the amount of the more valuable C₇ and C₈ alkylaromatics, specifically toluene and xylenes, which are produced in a dehydrocyclodimerization reaction zone. By way of example and not limitation, a suitable system for carrying out the processes described herein includes a moving bed radial flow multi-stage reactor such as is described in U.S. Pat. Nos. 3,652,231; 3,692,496; 3,706,536; 3685,963; 3,825,116; 3,839,196; 3,839,197; 3,854,887; 3,856,662; 3,918,930; 3,981,824; 4,094,814; 4,110,081; and 4,403,909. The systems that may be used in the present process may also include regeneration systems and various aspects of moving catalyst bed operations and equipment as described in these patents. This reactor system has been widely employed commercially for the reforming of naphtha. fractions. Its use has also been described for the dehydrogenation of light paraffins.

The reaction zone operates under light aliphatic aromatization and alkylation (of aromatics with aliphatic hydrocarbon) conditions. Therefore the reaction zone operating conditions promote both the formation of aromatics from light hydrocarbons such as C₂-C₈ paraffins, and naphthenes.

Conditions for aromatization of light hydrocarbons are known to favor low pressures and high temperatures. Hence for the dehydrocyclodimerization typical conditions are described in U.S. Pat. No. 4,642,402 A. The preferred metallic component is gallium as described in the previously cited U.S. Pat. No. 4,180,689. The balance of the catalyst can be composed of a refractory binder or matrix that is optionally utilized to facilitate fabrication, provide strength, and reduce costs. Suitable binders can include inorganic oxides, such as at least one of alumina, magnesia, zirconia, chromia, titania, boria, thoria, zinc oxide and silica. Suitable binders can include phosphate of aluminum, zircornium, chromium, titanium, boron, thorium, aluminum, zince, silicon, and the mixtures of thereof

Aromatization and alkylation conditions, according to the present subject matter, include temperatures ranging from about 350° C. to 650° C. In another approach, the aromatization and alkylation conditions may include a temperature between about 752° F. and 1328° F. (400° C. and 720° C.).

Aromatization and alkylation conditions according to the present example include pressures between 0.1 Psia to 500 Psia. In one approach, the aromatization and alkylation conditions may include pressures under 200 psia. The aromatization and alkylation conditions in another approach include a pressure between 5 Psia and 100 Psia. Without being limited by theory, hydrogen-producing aromatization reactions are normally favored by lower pressures and high temperatures, and accordingly in one approach conditions may include a pressure under about 70 psia at the outlet of the reaction zones rich in light aliphatic hydrocarbons.

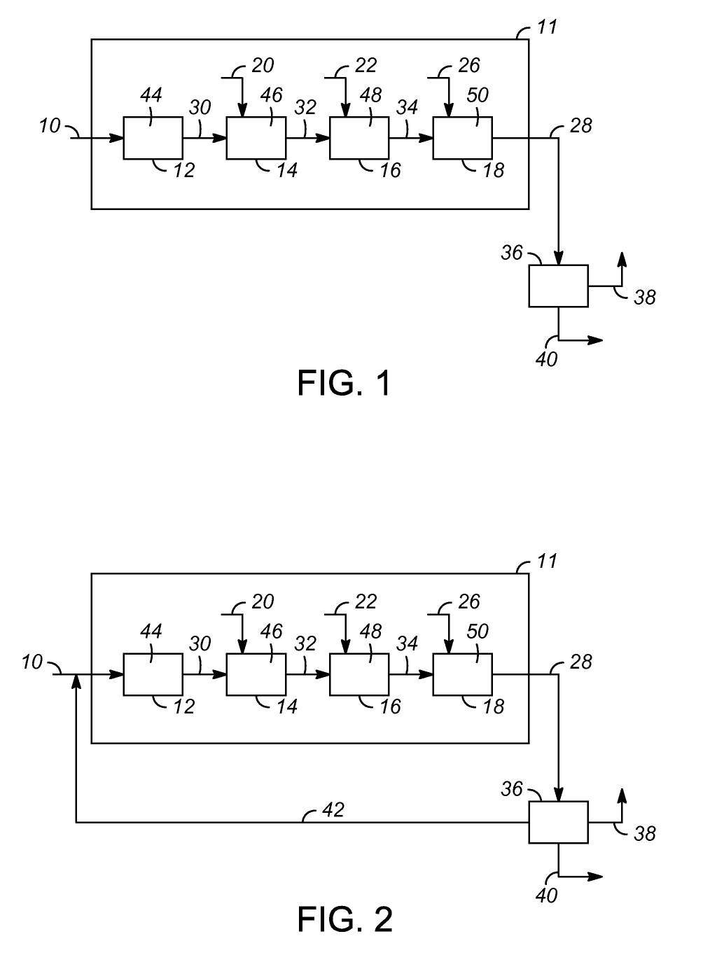

FIG. 1 illustrates a flow diagram of various embodiments of the processes described herein. Those skilled in the art will recognize that this process flow diagram has been simplified by the elimination of many pieces of process equipment including for example, heat exchangers, process control systems, pumps, fractionation column overhead and reboiler systems, etc. which are not necessary to an understanding of the process. It may also be readily discerned that the process flow presented in the drawing may be modified in many aspects without departing from the basic overall concept. For example, the depiction of required heat exchangers in the drawing have been held to a minimum for purposes of simplicity. Those skilled in the art will recognize that the choice of heat exchange methods employed to obtain the necessary heating and cooling at various points within the process is subject to a large amount of variation as to how it is performed. In a process as complex as this, there exists many possibilities for indirect heat exchange between different process streams. Depending on the specific location and circumstance of the installation of the subject process, it may also be desired to employ heat exchange against steam, hot oil, or process streams from other processing units not shown on the drawing.

FIG. 1 illustrates one example of a flow scheme illustrating the claimed subject matter. With reference to FIG. 1, a system and process in accordance with various embodiments includes a reaction zone 11. A feed stream 10 enters the reaction zone 11. The reaction zone 11 operates under typical aromatization of light hydrocarbon conditions in the presence of a typical aromatization of light hydrocarbon catalyst and produces a reaction zone product stream 28. The reaction zone 11 can include one or more reactor vessels that contain an aromatization catalyst. These reactors can further be connected with and without additional separation equipment, and they may be connected in series or in parallel. The reaction zone 11 may generate at least one outlet stream 28. The reaction zone outlet stream 28 may be sent to a separation zone 36.

In the example illustrated in FIG. 1, there are four reactors. However it is contemplated that there may be one or more reactors. The first reactor 12 contains a first catalyst 44. The feed stream 10 enters the first reactor 44, contacts the first catalyst 44 and forms a first reactor effluent 30. The first reactor effluent 30 and stream 20 then enter the second reactor 14, contact the second catalyst 46 and forms a second reactor effluent 32. The second reactor effluent 32 and stream 22 then enter the third reactor 16, contact the third catalyst 48 and forms a third reactor effluent 34. The third reactor effluent 34 and stream 26 enter the fourth reactor 18, contact the fourth catalyst 50 and form the reaction zone effluent 28.

As discussed previously, the feed stream 10 includes light aliphatic compounds. Light aliphatic compound streams can be introduced to the reaction zone 11 in a form that could be liquid, vapor, or a mixture thereof By way of one example, the fresh portion of a C₃ aliphatic feed may be available in liquid form as liquefied petroleum gas.

In one example, the feed stream 10 includes only C₃ rich hydrocarbons. Therefore, only C₃ rich hydrocarbons enter the first reactor 12. Streams 22 and 26 or streams 20, stream 22, and stream 26 include only C₄ rich hydrocarbons. Therefore, the C₄ rich hydrocarbons do not enter the first reactor 12, but the C₄ rich hydrocarbons only enter the second and third, or second, third, and fourth reactors. By feeding the less reactive C₃ rich feed into the first reactor 12 and the more reactive C₄ rich into the second reactors 14 and third reactor 16 or the second reactor 14, the third reactor 16, and the fourth reactor 18, a more desired aromatics yield results. This would also result in a reduced undesirable heavy aromatics, a reduced light ends including C₁ and C₂, and minimal pyrolytic coking in the lead reactor and heavy fouling in the lagging reactor, while maximizing C₃ conversions.

In this example, where a C₄ rich feed is introduced into lagging reactors, where both H₂ and aromatics are present, it greatly diverts the propensity to form butadiene, therefore reducing coke fouling. Furthermore, reducing contact times for C₄ conversions greatly mitigate the heavy aromatics formation, thus yields higher desirable aromatics products and mitigating the heavy fouling in the lag reactors. It is further recognized that introducing C₃ rich hydrocarbon into the lead reactor allows higher operating temperature and lower pressure to drive the conversion of less reactive C₃ rich hydrocarbon to form aromatics. This also minimizes the generation of coke and thus fouling, and minimizes the production of excessive light ends including C₁ and C₂, derived from the cracking of more reactive C₄.

In one example, the feed stream 10 includes only C₃ hydrocarbons. Therefore, only C₃ rich hydrocarbons enter the first reactor 12. Stream 20 and stream 22 include only C₄ rich hydrocarbons. Therefore, the C₄ rich hydrocarbons do not enter the first reactor 12, but the C₄ rich hydrocarbons only enter the second and third reactors. Stream 26 includes only C5 rich hydrocarbons.

In this embodiment, C₅ is introduced into the lag reactors to minimize and eliminate the high propensity to produce pyrolytic coke and heavy fouling in the lead and lag reactors. C₅ is a feed component in the dehydrocyclodimerization technology has difficulty processing at significant percentages in the overall feed.

In one example, the feed stream 10 includes only C₂ rich hydrocarbons. Therefore, only C₂ rich hydrocarbons enter the first reactor 12. Stream 20 includes only C₃ rich hydrocarbons. Therefore, the C₃ rich hydrocarbons do not enter the first reactor 12, but the C₃ rich hydrocarbons only enters the second reactor 14. Stream 22 and stream 26 include only C₄ rich hydrocarbons. Therefore the C₄ hydrocarbons only enter the third reactor 16 and the fourth reactor 18.

In this embodiment C₂, C₃ and C₄ rich hydrocarbons are introduced into reactors to attain descending contact times to maximize the overall aromatics yields with reducing light ends and heavy aromatics yields, while mitigating or eliminating pyrolytic coke and heavy fouling in the lead and lag reactor(s).

In another example, the feed stream 10 includes only C₂ rich hydrocarbons. Therefore, only C₂ rich hydrocarbons enter the first reactor 12. Stream 20 includes only C₃ rich hydrocarbons. Therefore, the C₃ rich hydrocarbons do not enter the first reactor 12, but the C₃ rich hydrocarbons only enters the second reactor 14. Stream 22 includes only C₄ rich hydrocarbons. Therefore the C₄ rich hydrocarbons only enter the third reactor 16. Stream 26 includes only C₅ rich hydrocarbons. Therefore a hydrocarbon stream rich in C₅ hydrocarbons only enters the fourth reactor 18.

FIG. 2 is similar to FIG. 1, however in FIG. 2, there is a recycle stream 42. The recycle stream contains C₂-C₄ hydrocarbons. The recycle stream 42 containing C₂-C₄ hydrocarbons may be mixed with the feed 10 as shown in FIG. 2, but the recycle stream 42 may also enter any or all of the reactors as well. For example, the recycle stream 42 may also enter the second reactor 14, the third reactor 16, and the fourth reactor 18.

As illustrated in FIG. 2, once the recycle stream 42 is combined with the feed 10, the feed 10 will contain whatever hydrocarbon is in the feed 10 plus the C₂-C₄ hydrocarbons present in the recycle stream 42. In one example, the feed stream 10 includes a hydrocarbon stream rich in C₃ hydrocarbons. Therefore, a hydrocarbon stream rich in C₃ hydrocarbons enters the first reactor 12. As used herein, the term “rich” can mean an amount of at least generally 50%, and preferably 70%, by mole, of a compound or class of compounds in a stream. Stream 20 and stream 22 include a hydrocarbon stream rich in C₄ hydrocarbons. Therefore, a hydrocarbon stream rich in C₄ hydrocarbons does not enter the first reactor 12, but a hydrocarbon stream rich in C₄ hydrocarbons only enters the second and third reactors. Stream 26 includes a hydrocarbon stream rich in C₅ hydrocarbons.

In one example, the feed stream 10 includes a hydrocarbon stream rich in C₂ hydrocarbons. Therefore, a hydrocarbon stream rich in C₂ hydrocarbons enter the first reactor 12. Stream 20 includes a hydrocarbon stream rich in C₃ hydrocarbons. Therefore, a hydrocarbon stream rich in C₃ hydrocarbons does not enter the first reactor 12, but the hydrocarbon stream rich in C₃ hydrocarbons only enters the second reactor 14. Stream 22 and stream 26 include a hydrocarbon stream rich in C₄ hydrocarbons or C₄ hydrocarbons and C₅ hydrocarbons respectively. Therefore a hydrocarbon stream rich in C₄ hydrocarbons enters the third reactor 16 and the fourth reactor 18 or C₄ hydrocarbons enters the third reactor 16 and C₅ hydrocarbons enters the forth reactor 18.

In this embodiment hydrocarbon streams rich in C₂, C₃, C₄, and C₅ are introduced into reactors to attain descending contact times to maximize the overall aromatics yields with reducing light ends and heavy aromatics yields, while mitigating or eliminating coke and heavy fouling in the lead and lag reactor(s).

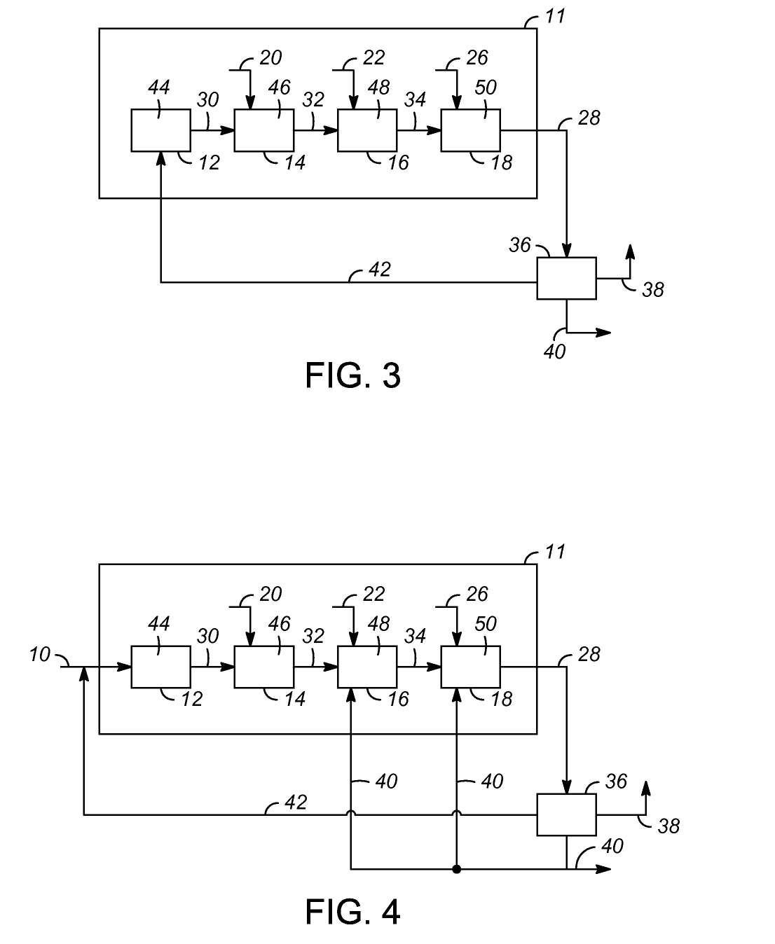

FIG. 3 is similar to FIG. 2, however in FIG. 3, the only feed entering the first reactor 12 is the recycle stream 42. Stream 20 includes a stream rich in C₃ hydrocarbons entering the second reactor 14. Stream 22 and stream 26 include hydrocarbon streams rich in C₄ hydrocarbons. Therefore a hydrocarbon stream rich in C₄ hydrocarbons enters the third reactor 16 and the fourth reactor 18.

In yet another example illustrated in FIG. 3, stream 20 includes a hydrocarbon stream rich in C₃ hydrocarbons entering the second reactor 14. Stream 22 includes a hydrocarbon stream rich in C₄ hydrocarbons. Therefore a hydrocarbon stream rich in C₄ hydrocarbons only enters the third reactor 16. Stream 26 includes a hydrocarbon stream rich in C₅ hydrocarbons. Therefore a hydrocarbon stream rich in C₅ hydrocarbons enters the fourth reactor 18.

Any suitable catalyst may be utilized such as at least one molecular sieve including any suitable material, e.g., alumino-silicate. The catalyst can include an effective amount of the molecular sieve, which can be a zeolite with at least one pore having a 10 or higher member ring structure and can have one or higher dimension. Typically, the zeolite can have a Si/Al₂ mole ratio of greater than 10:1, preferably 20:1-60:1. Preferred molecular sieves can include BEA, MTW, FAU (including zeolite Y in both cubic and hexagonal forms, and zeolite X), MOR, MSE, LTL, ITH, ITW, MFI, MEL, MFI/MEL intergrowth, TUN, IMF, FER, TON, MFS, IWW, EUO, MTT, HEU, CHA, ERI, MWW, AEL, AFO, ATO, and LTA. Preferably, the zeolite can be MFI, MEL, WI/MEL intergrowth, TUN, IMF, ITH and/or MTW. Suitable zeolite amounts in the catalyst may range from 1-100%, and preferably from 10-90%, by weight.

Generally, the aromatization and alkylation catalyst includes at least one metal selected from active metals, and optionally at least one metal selected from modifier metals, and the alkylation catalyst (of aromatic with paraffin) includes optionally no active metals. The total active metal content on the catalyst by weight is about less than 5% by weight. In some embodiments, the preferred total active metal content is less than about 2.5%, in yet in another embodiments the preferred total active metal content is less than 1.5%, still in yet in another embodiment the total active metal content on the catalyst by weight is less than 0.5 wt%. At least one metal is selected from IUPAC Groups that include 6, 7, 8, 9, 10, and 13. The IUPAC Group 7 trough 10 includes without limitation chromium, molybdenum, tungsten, rhenium, platinum, palladium, rhodium, iridium, ruthenium, osmium, silver, and zinc. The IUPAC Group 13 includes without limitation gallium and indium. In addition to at least one active metal, the catalyst may also contain at least one modifier metal selected from IUPAC Groups 11-17. The IUPAC Group 11 through 17 includes without limitation sulfur, gold, tin, germanium, and lead.

It is contemplated that the first catalyst 44, the second catalyst 46, the third catalyst 48, and the fourth catalyst 50 may be the same. However, it is also contemplated that the first catalyst 44, the second catalyst 46, the third catalyst 48, and the fourth catalyst 50 may be different.

In the example illustrated in FIG. 1, the reaction zone product stream 28 is sent to a light product separation zone 36 where one or more streams are generated. In this example, the light product separation zone 36 produces a first outlet stream 38, a second outlet stream 42, and a third outlet stream 40. The first light product separation zone outlet stream 38 contains hydrogen, C₁, and C₂ hydrocarbons. The second light product separation zone outlet stream 42 is rich in C₂-C₄ hydrocarbons, which may include a purge of the C₂-C₄ hydrocarbons, but also recycles the C₂-C₄ hydrocarbons to be mixed with the feed 10. The third light product separation zone outlet stream 40 contains C₆+ aromatics and is sent to the aromatic product separation zone. The light product separation zone 36 may have multiple separation vessels, each having multiple outlet streams comprising hydrogen, C₁-C₂ hydrocarbons, and C₂-C₄ hydrocarbons. These vessels may include but not limited to flash drums, condensers, reboilers, trayed or packed towers, distillation towers, adsorbers, cryogenic loops, compressors, and combinations thereof.

The recycle stream 42 containing C₂-C₄ hydrocarbons may be mixed with the feed 10 as discussed previously, but the recycle stream 42 may also enter any or all of the reactors as well. For example, the recycle stream 42 may also enter the second reactor 14, the third reactor 16, and the fourth reactor 18.

FIG. 4 illustrates yet another embodiment. In FIG. 4, the third light product separation zone outlet stream 40 containing C₆+ aromatics is sent to the aromatic product separation zone, but a portion of the outlet stream 40 is also sent to the fourth reactor 18, or the third reactor 16 and the fourth reactor 18. Stream 40 containing C6+ aromatics can be further separated and having selective aromatics such as xylene, toluene or preferably benzene and toluene or most preferably benzene sent to the fourth reactor 18 or the third reactor 16 and fourth reactor 18. In one embodiment the third reactor 16 and the fourth reactor 18 might have three streams entering each reactor. Therefore the aromatic rich product stream 40 is combined with the light aliphatic hydrocarbon stream to feed the third and fourth reactors containing the third and fourth catalyst, respectively. In another embodiment no light aliphatic hydrocarbons are introduced to the third reactor 16 or the fourth reactor 18. In this embodiment, the alkylation of unconverted light aliphatic hydrocarbon with aromatics is maximized and the amount of unconverted hydrocarbons in minimized. Consequently, recycling the unconverted light aliphatic hydrocarbons is minimized or eliminated entirely. In this embodiment C₂-C₃ rich feed enters the first reactor 12 and C₃-C₄ rich feed enters the second reactor 14 or the second reactor 14 and the third reactor 16.

It should be noted that various changes and modifications to the presently preferred embodiments described herein will be apparent to those skilled in the art. Such changes and modifications may be made without departing from the spirit and scope of the present subject matter and without diminishing its attendant advantages.

### SPECIFIC EMBODIMENTS

While the following is described in conjunction with specific embodiments, it will be understood that this description is intended to illustrate and not limit the scope of the preceding description and the appended claims.

A first embodiment of the invention is a process of producing aromatics hydrocarbons comprising passing a first light aliphatic hydrocarbon feed stream rich in at least C2 hydrocarbons, C3 hydrocarbons, or a combination thereof to a first reaction zone having a first catalyst to form a first reaction zone effluent; and passing the first reaction zone effluent and a second light aliphatic hydrocarbon feed stream rich in at least C3 hydrocarbons, C4 hydrocarbons, C5 hydrocarbons, or a combination thereof to second reaction zone comprising a second catalyst to form second reaction zone effluent. An embodiment of the invention is one, any or all of prior embodiments in this paragraph up through the first embodiment in this paragraph further comprising passing a third light aliphatic hydrocarbon feed stream into a third reaction zone comprising a third catalyst to form third reaction zone effluent. An embodiment of the invention is one, any or all of prior embodiments in this paragraph up through the first embodiment in this paragraph further comprising passing a fourth light aliphatic hydrocarbon feed stream into a fourth reaction zone comprising a fourth catalyst to form fourth reaction zone effluent. An embodiment of the invention is one, any or all of prior embodiments in this paragraph up through the first embodiment in this paragraph wherein the first light aliphatic hydrocarbon stream is rich in C3 hydrocarbons the second light aliphatic hydrocarbon stream is rich in C4 hydrocarbons. An embodiment of the invention is one, any or all of prior embodiments in this paragraph up through the first embodiment in this paragraph wherein the first light aliphatic hydrocarbon stream is rich in C2 hydrocarbons the second light aliphatic hydrocarbon stream is rich in C3 hydrocarbons. An embodiment of the invention is one, any or all of prior embodiments in this paragraph up through the first embodiment in this paragraph wherein the first light aliphatic hydrocarbon stream is rich in C2 hydrocarbons the second and third light aliphatic hydrocarbon stream is rich in C3 and C4 hydrocarbons. An embodiment of the invention is one, any or all of prior embodiments in this paragraph up through the first embodiment in this paragraph wherein the first light aliphatic hydrocarbon stream is rich in C2 hydrocarbons the second, third and subsequent light aliphatic hydrocarbon stream is rich in C3, C4, and C5 hydrocarbons. An embodiment of the invention is one, any or all of prior embodiments in this paragraph up through the first embodiment in this paragraph wherein the overall conversion of individual light hydrocarbon are within 30% and 99.5% conversions. An embodiment of the invention is one, any or all of prior embodiments in this paragraph up through the first embodiment in this paragraph wherein the overall conversions of individual light hydrocarbon are within 50% and 95% conversions. An embodiment of the invention is one, any or all of prior embodiments in this paragraph up through the first embodiment in this paragraph wherein the catalysts in the first and second reaction zones are the same catalyst and the process is fixed bed, moving bed or fluidized bed reactor. An embodiment of the invention is one, any or all of prior embodiments in this paragraph up through the first embodiment in this paragraph wherein the catalyst in the first and second reaction zones are different, and the process is fixed bed reactor. An embodiment of the invention is one, any or all of prior embodiments in this paragraph up through the first embodiment in this paragraph wherein a portion of light aliphatic hydrocarbon and heavy aromatics in the reactor effluent is separated from the aromatic product consisting of 6 to 10 carbon number with a single aromatic ring and the aromatic rich product stream is sent to the second reaction zone containing the second catalyst. An embodiment of the invention is one, any or all of prior embodiments in this paragraph up through the first embodiment in this paragraph wherein a portion of light aliphatic hydrocarbon in the reactor effluent is separated from the aromatic product and combined with the first light aliphatic hydrocarbon to feed the first reaction zone containing the first catalyst. An embodiment of the invention is one, any or all of prior embodiments in this paragraph up through the first embodiment in this paragraph wherein a portion of light aliphatic hydrocarbon consisting mostly C2, C3, and C4 in the reactor effluent is separated from the aromatic product and is fed to the first reaction zone containing the first reactor with the first light aliphatic hydrocarbon feeds to the second reaction zone containing the second reaction zone catalyst. An embodiment of the invention is one, any or all of prior embodiments in this paragraph up through the first embodiment in this paragraph wherein a portion of aromatic products in the reactor effluent is separated from the light aliphatic hydrocarbon and heavy aromatic hydrocarbon and combined with the second or third reaction zone effluent to feed to the third or fourth reaction zone containing third or fourth catalyst. The aromatic product in claim 13 is benzene, toluene, xylene, ethylbenzene, trimethylbenzene, methylethylbenzene, and preferably rich in benzene, toluene and xylene. An embodiment of the invention is one, any or all of prior embodiments in this paragraph up through the first embodiment in this paragraph, wherein the pressure of the first reaction zone is between about 0.1 to about 50 Psia and the temperature is from 400° C. to 850° C. An embodiment of the invention is one, any or all of prior embodiments in this paragraph up through the first embodiment in this paragraph, wherein the pressure of the second reaction zone is between about 1 Psia to about 500 Psia and the temperature is from 300° C. to 750° C. An embodiment of the invention is one, any or all of prior embodiments in this paragraph up through the first embodiment in this paragraph wherein the first catalyst and the second catalyst comprises a zeolite and at least one active metal-containing component. An embodiment of the invention is one, any or all of prior embodiments in this paragraph up through the first embodiment in this paragraph, wherein the second light aliphatic hydrocarbon feed stream is rich in hydrocarbons having a carbon number greater than the carbon number in the first light aliphatic hydrocarbon feed stream.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process of producing aromatics hydrocarbons comprising:
passing a first light aliphatic hydrocarbon feed stream rich in at least C₂ hydrocarbons, C₃ hydrocarbons, or a combination thereof to a first reaction zone having a first catalyst to form a first reaction zone effluent; and
passing the first reaction zone effluent and a second light aliphatic hydrocarbon feed stream rich in at least C₃ hydrocarbons, C₄ hydrocarbons, C₅ hydrocarbons, or a combination thereof to second reaction zone comprising a second catalyst to form second reaction zone effluent.

2. The method of claim 1 further comprising passing a third light aliphatic hydrocarbon feed stream into a third reaction zone comprising a third catalyst to form third reaction zone effluent.

3. The method of claim 2 further comprising passing a fourth light aliphatic hydrocarbon feed stream into a fourth reaction zone comprising a fourth catalyst to form fourth reaction zone effluent.

4. The method of claim 1 wherein the first light aliphatic hydrocarbon stream is rich in C₃ hydrocarbons the second light aliphatic hydrocarbon stream is rich in C₄ hydrocarbons.

5. The method of claim 1 wherein the first light aliphatic hydrocarbon stream is rich in C₂ hydrocarbons the second light aliphatic hydrocarbon stream is rich in C₃ hydrocarbons.

6. The method of claim 1 wherein the first light aliphatic hydrocarbon stream is rich in C₂ hydrocarbons the second and third light aliphatic hydrocarbon stream is rich in C₃ and C₄ hydrocarbons.

7. The method of claim 1 wherein the first light aliphatic hydrocarbon stream is rich in C₂ hydrocarbons the second, third and subsequent light aliphatic hydrocarbon stream is rich in C₃, C₄, and C₅ hydrocarbons.

8. The method of claim 1 wherein the overall conversion of individual light hydrocarbon are within 30% and 99.5% conversions.

9. The method of claim 1 wherein the overall conversions of individual light hydrocarbon are within 50% and 95% conversions.

10. The method of claim 1 wherein the catalysts in the first and second reaction zones are the same catalyst and the process is fixed bed, moving bed or fluidized bed reactor.

11. The method of claim 1 wherein the catalyst in the first and second reaction zones are different, and the process is fixed bed reactor.

12. The method of claim 1 wherein a portion of light aliphatic hydrocarbon and heavy aromatics in the reactor effluent is separated from the aromatic product consisting of 6 to 10 carbon number with a single aromatic ring and the aromatic rich product stream is sent to the second reaction zone containing the second catalyst.

13. The method of claim 1 wherein a portion of light aliphatic hydrocarbon in the reactor effluent is separated from the aromatic product and combined with the first light aliphatic hydrocarbon to feed the first reaction zone containing the first catalyst.

14. The method of claim 1 wherein a portion of light aliphatic hydrocarbon consisting mostly C₂, C₃, and C₄ in the reactor effluent is separated from the aromatic product and is fed to the first reaction zone containing the first reactor with the first light aliphatic hydrocarbon feeds to the second reaction zone containing the second reaction zone catalyst.

15. The method of claim 1 wherein a portion of aromatic products in the reactor effluent is separated from the light aliphatic hydrocarbon and heavy aromatic hydrocarbon and combined with the second or third reaction zone effluent to feed to the third or fourth reaction zone containing third or fourth catalyst.

16. The aromatic product in claim 13 is benzene, toluene, xylene, ethylbenzene, trimethylbenzene, methylethylbenzene, and preferably rich in benzene, toluene and xylene.

17. The method of claim 1, wherein the pressure of the first reaction zone is between about 0.1 to about 50 Psia and the temperature is from 400° C. to 850° C.

18. The method of claim 1, wherein the pressure of the second reaction zone is between about 1 Psia to about 500 Psia and the temperature is from 300° C. to 750° C.

19. The method of claim 1 wherein the first catalyst and the second catalyst comprises a zeolite and at least one active metal-containing component.

20. The method of claim 1, wherein the second light aliphatic hydrocarbon feed stream is rich in hydrocarbons having a carbon number greater than the carbon number in the first light aliphatic hydrocarbon feed stream.
